(19) United States
(12) Patent Application Publication
(10) Pub. No.: US 2005/0236976 A1
(43) Pub. Date: Oct. 27, 2005

(54) Organic electroluminescent device

(75) Inventors: Man-Kit Leung, Taipei City (TW); Hsien-Chang Lin, Chu-Pei City (TW); Meng-Yen Chou, Fengshan City (TW); Shen-Shen Wang, Taipei City (TW); Kuei-Hui Yang, Taipei City (TW)

Correspondence Address: BACON & THOMAS, PLLC, 625 SLATERS LANE, FOURTH FLOOR, ALEXANDRIA, VA, 22314, US

(73) Assignee: RiTdisplay Corporation, Hukou Township (TW)

(21) Appl. No.: 11/024,798

(22) Filed: Dec. 30, 2004

(30) Foreign Application Priority Data
Dec. 31, 2003 (TW) 092137789

(51) Int. Cl. H01J 1/62; H01J 63/04; C07D 43/02; C07C 211/54

(52) U.S. Cl. 313/504; 564/434; 548/440

(57) ABSTRACT

The present invention relates to an organic electroluminescent device, comprising a first electrode, an organic luminescent layer and a second electrode which disposed over a substrate. The organic electroluminescent layer comprises compound of formula (I) compound, wherein that Ar₁—Ar₆ are individual hydrogen, substituted or unsubstituted C₁₋₆ alkyl, substituted or unsubstituted C₃₋₆ cycloalkyl, substituted or unsubstituted C₃₋₁₀ alkenyl, substituted or unsubstituted C₆₋₄₀ aromatic amino, substituted or unsubstituted C₆₋₄₀ aromatic, substituted or unsubstituted C₆₋₄₀ poly cyclic aromatic, or substituted or unsubstituted C₆₋₄₀ aralkyl.

### BACKGROUND OF THE INVENTION

1. Field of the Invention

The present invention relates to an organic electroluminescent material.

2. Description of Related Art

Currently, lightweight and high efficient displays are widely developed, especially the liquid crystal display, LCD. However, LCD still has many shortcomings, such as narrow viewing angle, slow response time, difficulty for high-speed animation and the high electricity consumption of the back light module.

The organic electroluminescent device is provided with the self-luminescent property of organic electroluminescent material and the high-responding speed that can overcome the shortcomings mentioned above.

The organic electroluminescent device is consisting of an organic functional layer, the organic functional layer is provided or doped with luminescent compound. When electricity passes through transparent anode and metal cathode, the electron and the hole are recombined in the light-emitting layer and further generate exciton, which the light-emitting phenomenon above makes the material of the luminescent layer show luminescent property. The luminescent layer of different luminescent materials has different brightness or color, which is further based on the brand gap between ground state and exciting state of the material.

In practice, the luminescent brightness and efficiency of organic electroluminescent device usually decay in a short time. The main reason of the decay is causing by the damage of hole-transporting material. In order to overcome this problem, it has developed a hole-transporting material, which is provided with a high glass transition temperature (Tg), for example, N,N′-di(naphthyl-1-yl)-N′-diphenyl-4,4′-benzidine (NPB). The Tg of NPB is only 96° C., as a hole-transporting material, the lifetime to storage and operation at a high temperature is still short. Therefore, the thermal stability of NPB is not good enough for being a hole-transporting material.

Therefore, it is desirable to provide an improved material and device to mitigate and/or obviate the aforementioned problems.

### SUMMARY OF THE INVENTION

The present invention is to provide a hole-transporting material with a long-life and good thermal stability.

To achieve the object, the organic electroluminescent material of present invention includes a compound of formula (I): 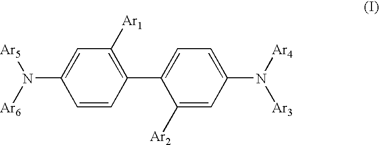
wherein Ar₁—Ar₆ are independently hydrogen, substituted or unsubstituted C₁₋₆ alkyl, substituted or unsubstituted C₃₋₆ cycloalkyl, substituted or unsubstituted C₃₋₁₀ alkenyl, substituted or unsubstituted C₆₋₄₀ aromatic amino, substituted or unsubstituted C₆₋₄₀ aromatic, substituted or unsubstituted C₆₋₄₀ poly cyclic aromatic, or substituted or unsubstituted C₆₋₄₀ aralkyl.

The organic electroluminescent material of present invention includes a compound of formula (I) can be used in any layer of the organic functional layer, preferably for the material of hole-transporting layer. The luminescent compound of the present invention, alternatively the substituted or unsubstituted of C₁₋₆ alkyl is preferred to be methyl, ethyl, propyl, isopropyl, butyl, isobutene, sec-butyl, tert-butyl, pentyl, or hexyl. The aromatic is provided with optional substituents. Preferably, the substituent on the aromatic group is alkyl of 1-6 carbon, cycloalkyl of 3-6 carbon, alkoxy of 1-6 carbon, aryloxy of 5-18 carbon, aralkoxy of 7-18 carbon, amino substituted with aromatic substituent consisting of 5-16 carbon, nitro, cyano and ester consisting of 1-6 carbon, or halogen. Among them, the cycloakoxy consisting 3-6 carbon is, preferably, cyclopropyl, cyclobutyl, cyclopentyl or cyclohexyl. The alkoxy consisting of 1-6 carbon is preferred to be methoxy, ethoxy, propoxy, isopropoxy, butoxy, isobutoxy, sec-butoxy, tert-butoxy, various isopropoxy or various hexoxyl. The aryloxy consisting of 5-18 carbon is preferred to be phenoxy, tolyoxymethyl or napthoxy. The aralkoxy consisting of 7-18 carbon, preferably, is phenyl ethoxyl or naphthymethoxyl. The amino substituted with substutient of aromatic consisting of 5-16 carbon is preferred to be diphenylamino, dimethylbenzeneamino or naphthylphenylamino. The ester consisting of 1-6 carbon is, more preferably, methoxycarbonyl, ethoxycarbonyl or propoxycarbonyl. The halogen illustrated above is not limited. More preferably, the halogen is fluorine, chlorine or bromine. The aromatic consisting of 6-40 carbon illustrated above is not limited. More preferably, the aromatic consisting of 6-40 carbon is phenyl, naphthyl, anthryl, phenanthryl, pyrenyl, biphenyl, terphenyl, triphenylamine, furan, thiophene or indole.

An organic electroluminescent material of the present invention is more preferred to be selected from the group consisting of:

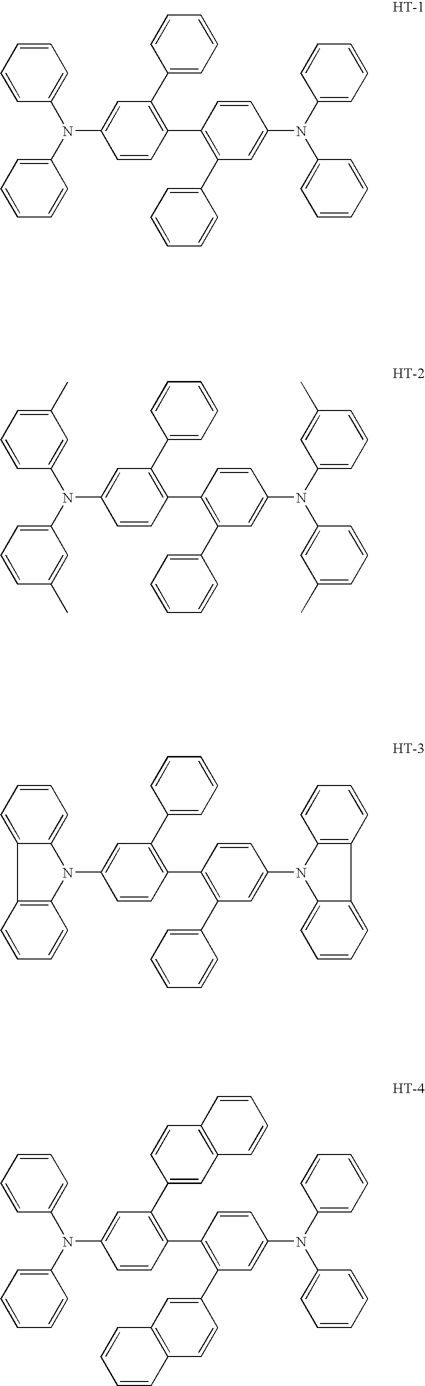

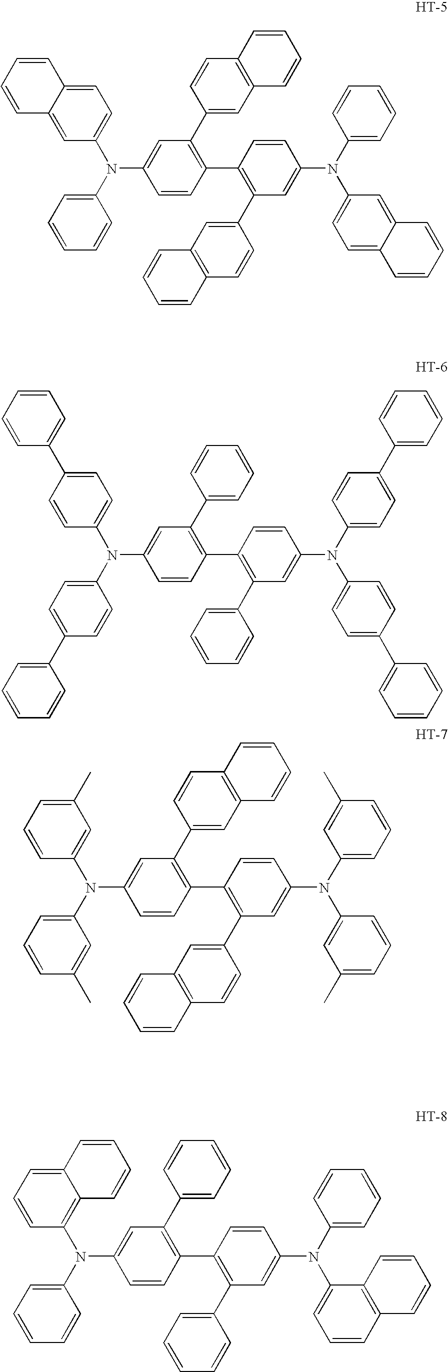

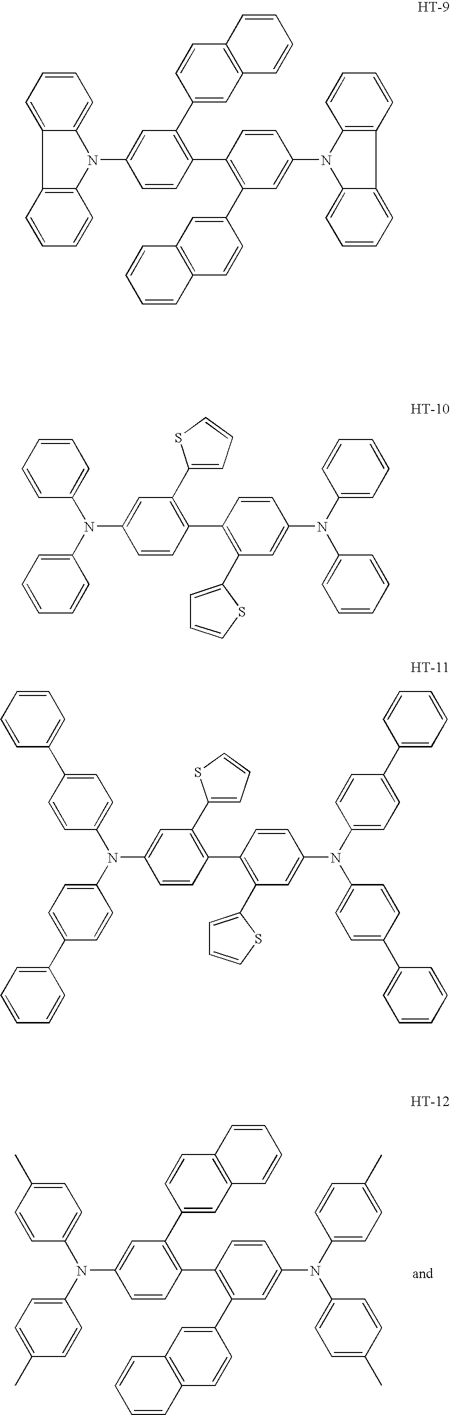

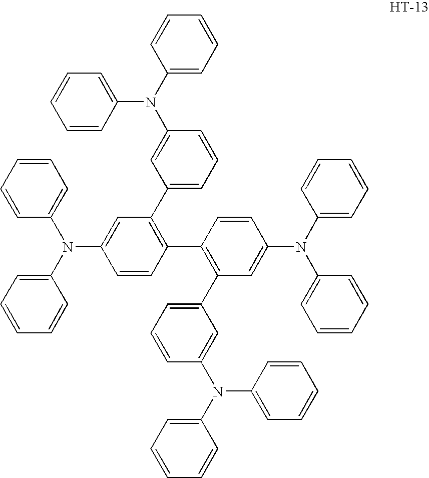

An organic electroluminescent device of the present invention comprising: at least a first electrode, an organic functional layer and a second electrode disposed over a substrate; the organic functional layer comprises the following compound of formula (I): 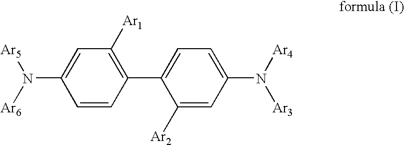
wherein Ar₁—Ar₆ are independently hydrogen, substituted or unsubstituted C₁₋₆ alkyl, substituted or unsubstituted C₃₋₆ cycloalkyl, substituted or unsubstituted C₃₋₁₀ alkenyl, substituted or unsubstituted C₆₋₄₀ aromatic amino, substituted or unsubstituted C₆₋₄₀ aromatic, substituted or unsubstituted C₆₋₄₀ poly cyclic aromatic, or substituted or unsubstituted C₆₋₄₀ aralkyl.

The organic electroluminescent device of present invention includes a luminescent of compound formula (I) which can be applied in any layer of the organic functional layer, preferably for the material of hole-transporting layer. The luminescent compound of the present invention, alternatively the substituted or unsubstituted of C₁₋₆ alkyl is preferred to be methyl, ethyl, propyl, isopropyl, butyl, isobutene, sec-butyl, tert-butyl, pentyl, or hexyl. The aromatic is provided with optional substituents. Preferably, the substituent on the aromatic group is alkyl of 1-6 carbon, cycloalkyl of 3-6 carbon, alkoxy of 1-6 carbon, aryloxy of 5-18 carbon, aralkoxy of 7-18 carbon, amino substituted with aromatic substituent consisting of 5-16 carbon, nitro, cyano and ester consisting of 1-6 carbon, or halogens. Among them, the cycloakoxy consisting 3-6 carbon is, preferably, cyclopropyl, cyclobutyl, cyclopentyl or cyclohexyl. The alkoxy consisting of 1-6 carbon is preferred to be methoxy, ethoxy, propoxy, isopropoxy, butoxy, isobutoxy, sec-butoxy, tert-butoxy, various isopropoxy or various hexoxyl. The aryloxy consisting of 5-18 carbon is preferred to be phenoxy, tolyoxymethyl or napthoxy. The aralkoxy consisting of 7-18 carbon, preferably, is phenyl ethoxyl or naphthymethoxyl. The amino substituted with substutient of aromatic consisting of 5-16 carbon is preferred to be diphenylamino, dimethylbenzeneamino or naphthylphenylamino. The ester consisting of 1-6 carbon is, more preferably, methoxycarbonyl, ethoxycarbonyl or propoxycarbonyl. The halogen illustrated above is not limited. More preferably, the halogen is fluorine, chlorine or bromine. The aromatic consisting of 6-40 carbon illustrated above is not limited. More preferably, the aromatic consisting of 6-40 carbon is phenyl, naphthyl, anthryl, phenanthryl, pyrenyl, biphenyl, terphenyl, triphenylamine, furan, thiophene or indole.

An organic electroluminescent device of the present invention, wherein the material is preferred to be selected from the group consisting of

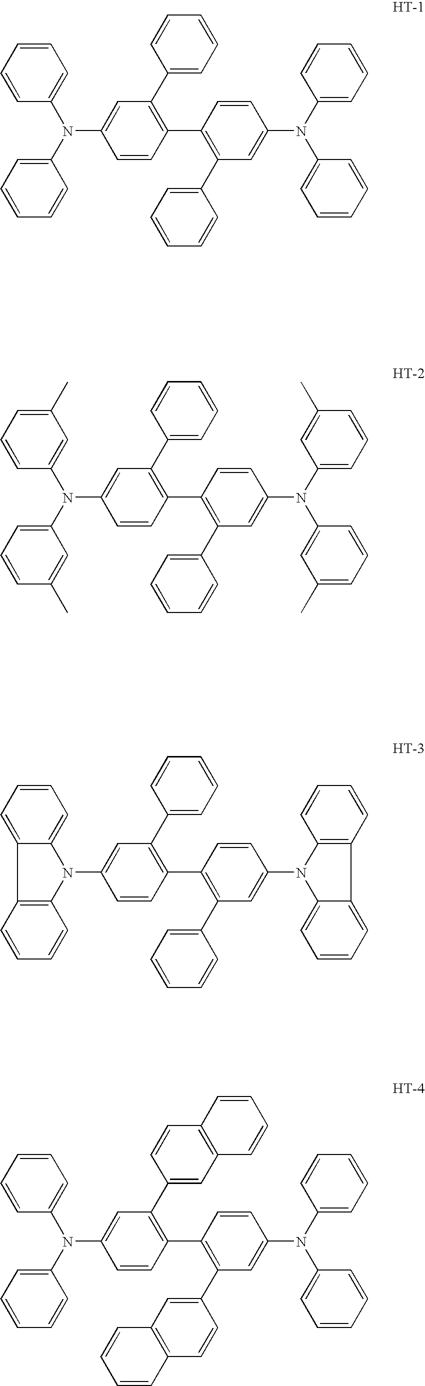

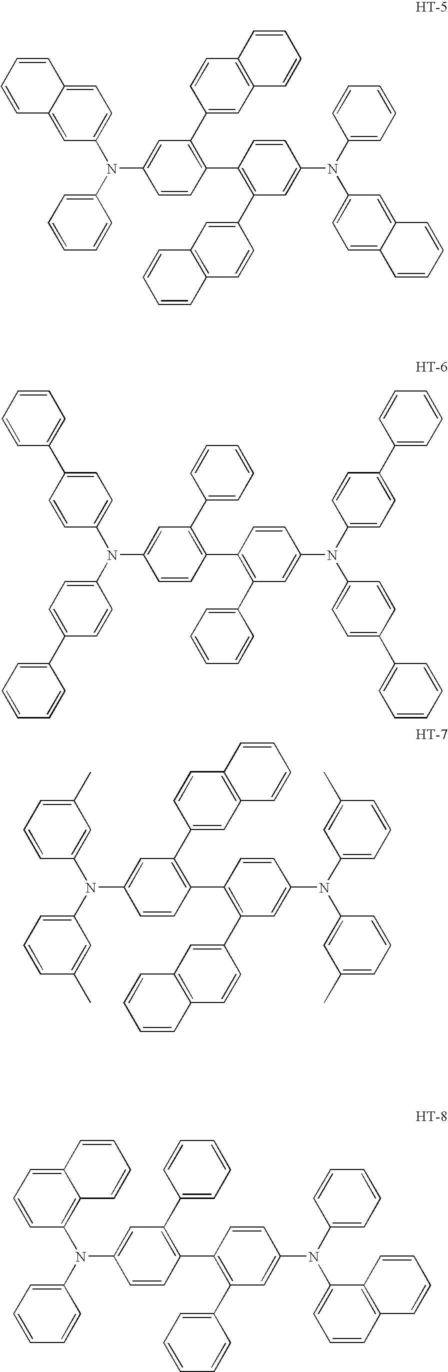

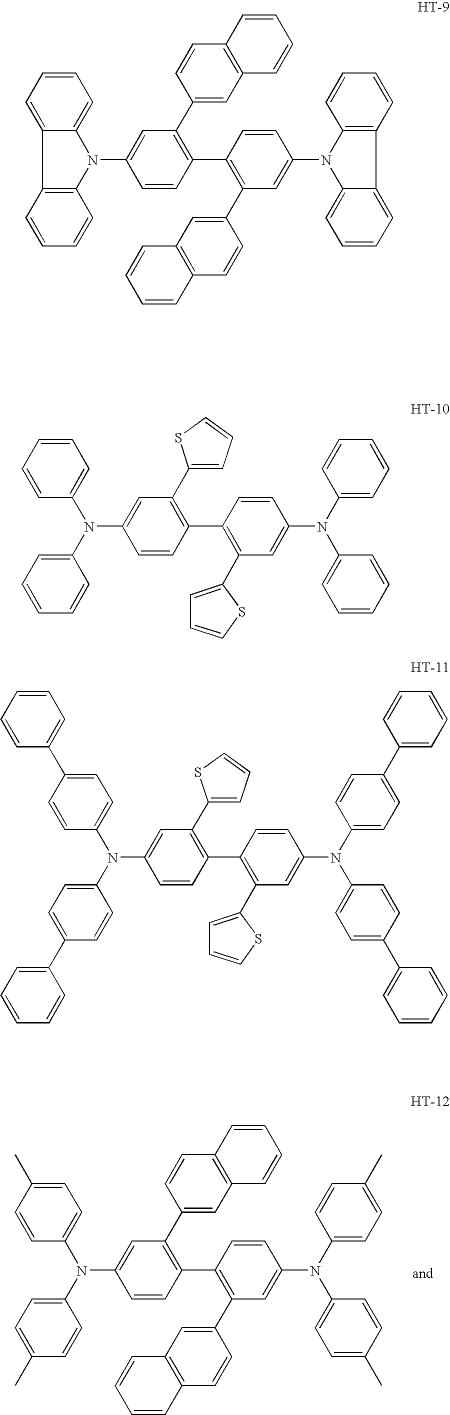

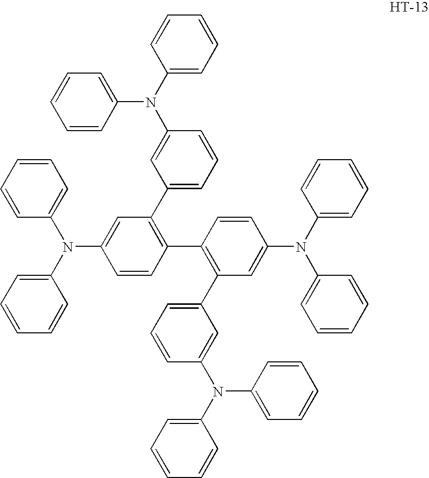

The organic functional layer of the organic electroluminescent device can be a single layer, double layers, or multi-layered laminate. The materials in the double layer, the single layer, or the multi-layered laminate can be materials having light-emitting capability, hole-transporting capability and electron-transporting capability. The method for forming various organic functional layer of the organic electroluminescent device is not limited. Preferably, the method is vacuum deposition, spin-coating, etc. the method for forming the organic functional layer which comprises compounds of formula (I) is not limited. The preferred methods are vacuum deposition, MBE, dip coating, spin coating, casting, bar code, or roll coating. There is no specific limitation for the thickness of various layer of the organic functional layer. Generally, pin holes or other defaults will form if the layer of the organic functional layer is too thin. On the other hand, thick organic functional layer will claim higher voltage then further to lower efficiency of the system. Therefore, the preferred thickness of layer of the organic functional layer is between 1 nm to 1 μm.

The organic electroluminescent device of present invention, wherein the organic functional layer can be a single layer or multi-layered laminate places in between first electrode and second electrode. Preferably, the structure of the multi-layered laminate is not limited. The preferred multi-layered laminate comprises hole-injectioning layer, hole-transporting layer, luminescence layer, electron-injectioning layer and the combination thereof. The sequence of the layers of the organic functional layer, the layered number of the organic functional layer is not limited. The preferred combination can be anode/luminescent layer/cathode, anode/luminescent layer/electron-transporting layer/cathode, anode/hole-transporting layer/luminescent layer/cathode, anode/hole-transporting layer/luminescent layer/electron-transporting layer/cathode, anode/hole-injectioning layer/hole-transporting layer/luminescent layer/cathode, anode/hole-injectioning layer/hole-transporting layer/luminescent layer/electron-transporting layer/cathode or anode/hole-injectioning layer/hole-transporting layer/luminescent layer/electron-injectioning layer/cathode.

Other objects, advantages, and novel features of the invention will become more apparent from the following detailed description when taken in conjunction with the accompanying drawings.

### BRIEF DESCRIPTION OF THE DRAWINGS

FIG. 1 is the organic luminescent device of present invention.

FIG. 2 is EL spectrum result of the present invention.

FIG. 3 is B-V (brightness-voltage) of the present invention.

### DETAILED DESCRIPTION OF THE PREFERRED EMBODIMENT

### EXAMPLE 1

### Synthesis of HT-1

The synthesis of HT-1 compound is completed by achieving the synthesis of compound A, and compound B first and then finally synthesizing HT-1 through coupling compound A and compound B. The synthesis of compound in the present invention is illustrated below:

### Compound A: 4,4′-Dinitro-biphenyl

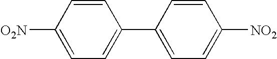

4.4 ml (65% HNO₃) and 5.2 ml (98% H₂SO₄) are added into a two-neck bottle, stirred in 0° C. ice bath. Then biphenyl (3 g, 19.5 mmol, 2M in MeNO₂) is slowly added into the mixed acid, then stirred for one hour in 0° C. ice bath, the mixture is heated to 35° C. then stirred for 2 hours. until the reaction is completed, The solution is poured into ice water, and the product and sub-product are filtered by vacuum filtration. The solid residue is washed by water, and the solid residue is collected. Then the solid residue heated and stirred in methanol, and is collected by vacuum filtration. After heating and stirring in toluene, the solid residue is collected by vacuum filtration. The light yellow solid is obtained, which is compound A mentioned above (0.825 g, 17%).

### Spectrum Reading Data

1H NMR (400 MHz, CDCl3): δ 8.34 (d, J=8.6 Hz, 4H), 7.77 (d, J=8.6 Hz, 4H); ¹³C NMR (100 MHz, CDCl₃): δ 148.06, 144.97, 128.32, 124.37. HRMS-FAB m/z calcd for C₁₂H₈NO₄ 244.0484, found 244.0497.

### Compound B: 2,2′-Diiodo2,2′-dinitrobiphenyl

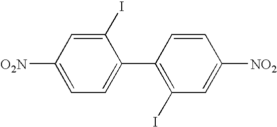

Compound A (10 g, 41 mmol), Ag₂SO₄ (34.3 g, 110 mmol) and iodine (31.2 g, 123 mmol) are added into a two-neck bottle installed with condenser The mixture is stirred for 16-48 hours under 120° C. The reaction is quenched according to the result of NMR spectroscopy, when the volume of compound is over 80%. The solution is poured ice Na₂S₂O₃.5H₂O solution; and the solid residue is collected by vacuum filtration, washing for several times. The solid is re-crystallized by ethyl acetate; and the yellow product is obtained as compound B (13.2 g, 65%).

### Spectrum Reading Data

mp 148-149° C.; 1H NMR (400 MHz, CDCl₃) 5 8.75 (d, J=2.2 Hz, 2H), 8.30 (dd, J=8.4, 2.2 Hz, 2H), 7.37 (d, J=2.2 Hz, 2H); ¹³C NMR (100 MHz, CDCl₃): δ 152.96, 147.60, 133.91, 129.84, 123.27, 97.91. HRMS-FAB m/z calcd for C₁₂H₆O₄N₂I₂ 495.8417, found 495.8410.

### Compound C: 4′,4″-Dinitro-[1,2′;1′,1″;2″,1′″]quaterphenyl

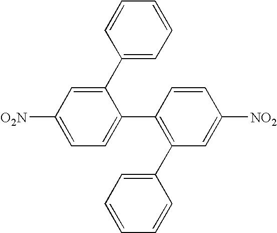

Compound B (5 g, 10 mmol), Pd (PPh₃)₄ (0.6 g, 0.5 mmol) and Na₂CO₃ (6.4 g, 60 mmol) are added into two-neck bottle installed with condenser. Then 25 ml benzene, 10 ml distilled water and 2 ml ethanol are added, stirred for 2 hours in 80° C. until the reaction completed. The solid obtained is washed with water and purified by chloroform. The chloroform solution is collected. The mixture is dried by MgSO₄, and the solution is filtered. The filtrate is obtained then condensed by the circumrotate decompressing condenser, and the product is then re-crystallized by CH₃NO₂. The light yellow solid product C is obtained (2 g, 50%).

### Spectrum Reading Data

mp 228-229° C.; 1H NMR (400 MHz, CDCl3): δ 8.27 (dd, J=8.5, 2.4 Hz, 2H), 8.11 (d, J=2.4 Hz, 2H), 7.62 (d, J=8.5 Hz, 2H), 7.24-7.10 (m, 6H), 6.64-6.62 (m, 4H); 13C NMR (100 MHz, CDCl3): δ 147.84, 144.49, 142.55, 137.89, 132.28, 128.89, 128.21, 127.66, 125.18, 122.04. HRMS-FAB m/z calcd for C24H16O4N2 396.1120, found 397.1170.

### Compound D: [1,2′;1′,1″;2″,1′″]Quaterphenyl-4′,4″-diamine

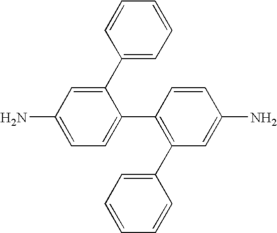

Ammonia formate (1.97 g, 30 mmol), 10% Pd/C (0.15 g, 1.4 mmol) and compound C (1.0 g, 2.5 mmol) are added into a two-neck bottle installed with condenser, 5 ml DMF is added then heated under 80° C. for 3 hours until the reaction is completed. The mixture is washed and purified by water and ethyl acetate, then dried by MgSO₄ and filtered The filtrate is collected and condensed by circumrotate decompressing condenser. Then purifying the solid again by column chromatography (wash reagent:ethyl acetate/CH3(CH2)CH3 =2/3), light yellow product D is obtained (0.66 g, 78%).

### Spectrum Reading Data

1H NMR (400 MHz, CD3COCD3): δ 7.05-6.95 (m, 10H), 6.71-6.68 (m, 4H), 6.61 (d, J=8.1 Hz, 2H), 6.47 (d, J=2.4 Hz, 2H), 4.54 (s, 4H); 13C NMR (100 MHz, CD3COCD3): δ 147.88, 143.25, 142.45, 133.40, 129.99, 1290.87, 127.98, 126.24, 116.58, 114.16. HRMS-FAB m/z calcd for C24H20N2 336.1627, found 336.1624.

### Compound HT-1

### N4′,N4′,N4″,N4″-Tetraphenyl-[1,2′;1′,1″;2″,1′″]quaterphenyl-4′,4″-diamine

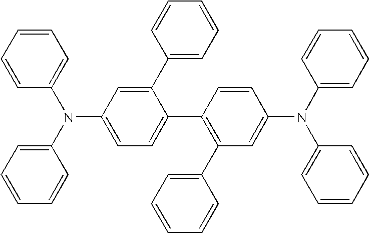

Compound D (0.15 g, 0.44 mmol), palladium acetate (2.5 mg, 0.0089 mmol), DPPF (7.7 mg, 0.0134 mmol) and sodium tert-butaoxide (0.23 g, 2.23 mmol) are added into a two-neck bottle installed with condenser, then 2.2 ml toluene and bromobenzene (C₆H₅Br, 0.38 ml, 3.57 mmol) are added, the solution is heated to 110° C. and refluxed under argon gas for 18 hours until the reaction completed, washed and purified by water and ethyl acetate, the ethyl acetate solution is collected, dryed by MgSO4. The filtrate is condensed by circumrotate decompressing condenser, then the product is purified again by column chromatography(wash reagent:ethyl acetate/CH3(CH2)CH3=2/75). A white solid product HT-1 (0.2 g, 70%) is obtained.

### Spectrum Reading Data

1H NMR (400 MHz, CDCl3): δ 7.34 (d, J=8.3 Hz, 2H), 7.27 (t, J=7.5 Hz, 8H), 7.13 (d, J=7.6 Hz, 8H), 7.08 (m, 4H), 7.04-7.01 (m, 8H), 6.94 (d, J=2.3 Hz, 2H); 13C NMR (100 MHz, CDCl3): δ 147.73, 146.84, 142.08, 140.71, 134.40, 132.34, 129.24, 129.18, 127.21, 126.00, 125.48, 124.01, 122.82, 122.62. HRMS-FAB m/z calcd for C48H36N2 640.2879, found 640.2873. Anal. calcd for C48H36N2: C, 89.97; H, 5.66; N, 4.37, found C, 89.64; H, 5.69; N, 4.16.

### EXAMPLE 2

### Synthesis of HT-4

### Compound E: (2,2′-Dinaphthalen-1-yl-4,4′-dinitrobiphenyl)

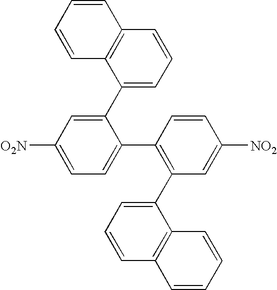

Compound B (10.0 g, 20 mmol), 1-naphthalene boric acid (20.8 g, 121 mmol), Pd(PPh3)4 (0.46 g, 0.4 mmol) and (6.6 g, 62 mmol) are added into a two-neck bottle installed with condenser, then 30 ml benzene, 15 ml water and 5 ml ethanol are added and stirred for 3 hours under 80° C. in nitrogen gas until the reaction completed, the solution is washed and purified by water and chloroform; the chloroform solution is collected, and then dryed by MgSO4, the solution is filtered and the filtrate is condensed by the circumrotate decompressing condenser; the obtained solid is added into ethanol and stirred the solution, most of boric acid is removed; and the solid is collected by vacuum filtration and purified by column chromatography(wash reagent: ethyl acetate/CH3(CH2)CH3=1/6), yellow sold compound E(8.7 g, 87%) is obtained.

### Spectrum Reading Data

mp 203-204° C.; ¹H NMR (400 MHz, CDCl₃): δ 8.32-5.83 (m, 20H); ¹³C NMR (100 MHz, CDCl₃): δ 146.87, 145.89, 140.38, 135.14, 133.61, 132.64, 128.67, 128.25, 126.85, 126.65, 126.15, 125.83, 125.03, 124.41, 121.77. HRMS-FAB m/z calcd for C₃₂H₂₀O₄N₂ 496.1423, found 496.1419. Anal. calcd for C₃₂H₂₀O₄N₂: C, 77.41; H, 4.06; N, 5.64, found C, 77.36; H, 3.70; N, 5.44.

### Compound F: 2,2′-Di-naphthalen-1-yl-4,4′-diamine-biphenyl

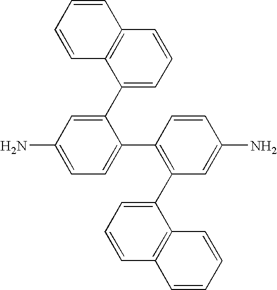

Ammonia formate (0.83 g, 7.8 mmol), 10% Pd/C (0.18 g, 2.8 mmol) and compound E (0.65 g, 1.3 mmol) are added into a two-neck bottle, then 2.6 ml DMF is added and the mixture is heated to 80° C. for 3 hours until the reaction completed, the solution is washed and purified by water and ethyl acetate; the ethyl acetate solution is collected and dryed by MgSO4, then the solution is filtered then the filtrate is condensed by circumrotate decompressing condenser and the obtained solid is purified by column chromatography (wash reagent: ethyl acetate/CH3(CH2)CH3=1), light yellow sold compound F(0.45 g, 73%) is obtained.

### Spectrum Reading Data

¹H NMR (400 MHz, CD₃COCD₃): δ 7.81-6.33 (m, 20H), 4.44 (s, 4H); ¹³C NMR (100 MHz, CD₃COCD₃): δ 146.72, 141.04, 140.40, 134.60, 133.70, 131.20, 128.56, 127.77, 127.36, 127.30, 125.91, 125.86, 125.33, 118.04, 117.87, 113.99. HRMS-FAB m/z calcd for C₃₂H₂₄N₂ 436.1940, found 436.1935. Anal. calcd for C₃₂H₂₄N₂: C, 88.04; H, 5.54; N, 6.42, C, 87.65; H, 5.56; N, 6.34.

### HT-4

### 2,2′-Di-naphthalen-1-yl-N4,N4,N4′,N4′-tetraphenyl-biphenyl-4,4′-diamine

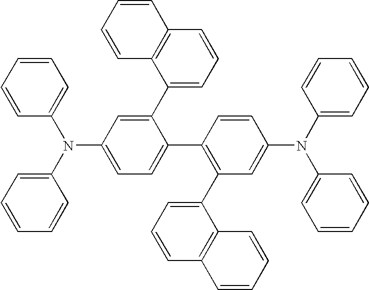

Compound F (1.69 g, 3.87 mmol), palladium acetate (42 mg, 0.19 mmol), DPPF (109 mg, 0.20 mmol) and Sodium tert-butaoxide (5.3 g, 2.23 mmol) are added into a two-neck bottle installed with condenser, then 16 ml toluene, bromobenzene (C₆H₅Br, 5 ml, 47 mmol) are added. The solution is heated to 110° C. and refluxed under argon gas for 18 hours until the reaction completed, then the mixture is washed and purified by water and ethyl acetate; the ethyl acetate solution is collected and dryed by MgSO4,the filtrate is condensed by circumrotate decompressing condenser, the product then purified again by column chromatography (wash reagent: ethyl acetate/CH₃(CH₂)CH₃=1/9) a white solid product HT-4 (1.7 g, 60%) is obtained.

### Spectrum Reading Data

¹H NMR (400 MHz, CDCl₃): δ 7.70-6.78 (m, 46H); ¹³C NMR (100 MHz, CDCl₃): δ 147.59, 145.80, 139.83, 135.84, 135.56, 133.54, 132.99, 129.09, 127.54, 127.27, 127.23, 127.05, 125.02, 124.97, 124.27, 123.99, 123.93, 122.68, 122.57 122.52. HRMS-FAB m/z calcd for C₅₆H₄₀N₂ 740.3192, found 740.3212. Anal. Calcd for C₅₆H₄₀N₂: C, 90.78; H, 5.44; N, 3.78, found C, 90.90; H, 5.32; N, 3.49.

### EXAMPLE 3

### Synthesis of HT-13

### Compound G: 4′,4″-Dinitro-N4,N4,N4′″,N4′″-tetraphenyl-[1,2′;1′,1″;2″,1′″]quaterphenyl-4,4′″-diamine

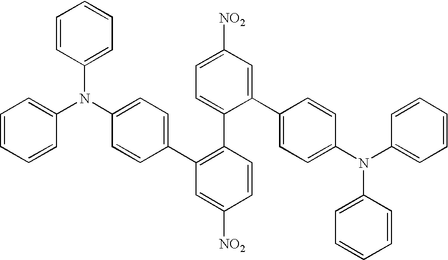

Compound B (2.6 g, 5.2 mmol), diphenylamine-phenylborate (11.57 g, 40 mmol), Pd(PPh₃)₄ (0.6 g, 0.52 mmol)and Na₂CO₃ (5.8 g, 54 mmol) are added into a two-neck bottle installed with condenser, then 26 ml benzene, 15 ml water and 6 ml ethanol are added and stirred for 6 hours under 80° C. in nitrogen gas until the reaction completed, the solution is washed and purified by water and chloroform; the chloroform solution is collected and dryed by MgSO₄, the solution is filtered, the filtrate is condensed by circumrotate decompressing condenser and purified again by column chromatography(wash reagent: ethyl acetate/CH₃(CH₂)CH_{3=1/5}), red brown sold compound F (2.1 g, 55%) is obtained.

### Spectrum Reading Data

¹H NMR (400 MHz, CDCl₃) δ 8.20 (dd, J=8.4, 2.3 Hz, 2H), 8.12 (d, J=2.3 Hz, 2H), 7.57 (d, J=8.4 Hz, ²H), 7.18 (t, J=8.1 Hz, 8H), 7.03-6.97 (m, 12H), 6.76 (d, J=8.6 Hz, 4H), 6.52 (d, J=8.6 Hz, 6H); ¹³C NMR (100 MHz, CDCl₃) δ 147.93, 147.59, 147.07, 144.46, 142.04, 132.20, 131.18, 129.52, 129.35, 124.79, 124.72, 123.50, 122.09, 121.71. HRMS-FAB m/z 730.2587, calcd for C₄₈H₃₄O₄N₄ 730.2580.

### Compound H

### N4,N4,N4′″,N4′″-Tetraphenyl-[1,2′;1′,1″;2″,1′″]quaterphenyl-4,4′,4″,4′″-tetra amine

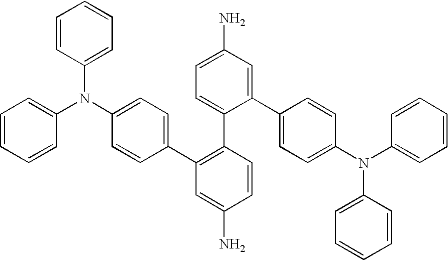

Compound G(7.83 g, 10.7 mmol) and SnCl₂.3H₂O (27.68 g, 114 mmol) are added into a 250 ml two-neck bottle, then 50 ml ethyl acetate and 50 ml ethanol are added and heated under 90° C. for 4 hours until the reaction completed then cool the solution, iced NaOH solution is added then the solution is washed by ethyl acetate and the water is keeping for layer use, the ethyl acetate layer is collected and dryed by MgSO₄, filtering the solution and condensing the filtrate by circumrotate decompressing condenser; the solid is purified again by column chromatography(wash reagent: ethyl acetate/CH₃(CH₂)CH₃=2/1), white sold compound H (4.72 g, 66%)is obtained.

### Spectrum Reading Data

¹H NMR (400 MHz, CDCl₃) δ 7.17-7.12 (m, 12H), 7.02-6.99 (m, 8H), 6.91 (t, J=7.3 Hz, 4H), 6.72 (d, J=8.7 Hz, 4H), 6.64 (dd, J=8.1, 2.5 Hz, 2H), 6.57 (d, J=8.7 Hz, 4H), 6.52 (d, J=2.4 Hz, 2H), 3.61 (s, 4H); ¹³C NMR (100 MHz, CDCl₃) δ 147.73, 145.61, 145.26, 141.22, 136.04, 132.71, 130.76, 129.68, 129.07, 123.95, 123.09, 122.48, 116.28, 114.02. HRMS-FAB m/z 670.3103, calcd for C₄₈H₃₈N₄ 670.3096.

### Compound HT-13

### N4,N4,N4′,N4′,N4″,N4″,N4′″,N4′″-Octaphenyl-[1,2′; 1′,1″;2″,1′″]quaterphen yl-4,4′,4″,4′″-tetraamine

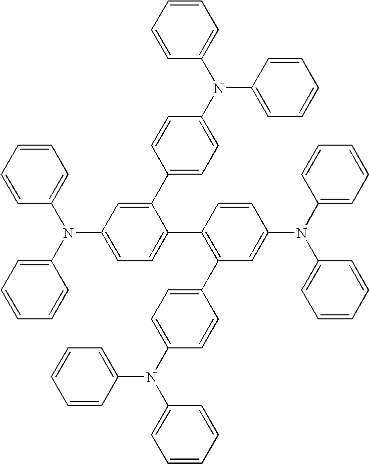

Compound H (1.21 g, 1.8 mmol), Pd₂ (dba) 3 (0.2 g, 0.22 mmol), DPPF (0.21 g, 0.38 mmol) and Sodium tert-butaoxide (1.81 g, 18.6 mmol) are added into a two-neck bottle installed with condenser, then 4 ml toluenebromobenzene (C₆H₅Br, 1.8 ml, 17 mmol) are added and the solution is heated to 100° C., refluxed under argon gas for 24 hours until the reaction completed, washed and purified by water and chloroform; the chloroform solution is collected and dryed by MgSO4₄, the filtrate is condensed by circumrotate decompressing condenser, then the product is purified again by column chromatography (wash reagent: ethyl acetate/ CH3(CH2)CH3=8/1), the obtained solid further mix with ethyl acetate and acetonitrile, a white solid product HT-13 (1.3 g, 74%) is obtained.

### Spectrum Reading Data

¹H NMR (400 MHz, CDCl₃) δ 7.31 (d, J=8.2 Hz, 2H), 7.22-6.92 (m, 44H), 6.71 (d, J=8.6 Hz, 4H), 6.50 (d, J=8.6 Hz, 4H); ¹³C NMR (100 MHz, CDCl₃) δ 147.75, 147.68, 146.94, 146.02, 141.43, 135.14, 132.29, 129.93, 129.91, 129.18, 125.16, 124.04, 124.01, 122.87, 122.73, 122.66, 122.61. HRMS-FAB m/z 974.4348, calcd for C₇₂H₅₄N₄ 974.4348. Anal. Calcd for C₇₂H₅₄N₄: C, 88.67; H, 5.58; N, 5.75. Found: C, 88.56; H, 5.64; N, 5.76.

### EXAMPLE 4

### Preparing Method of the Device

The example is mainly used for illustrating the function of the organic electroluminescent device structure with the organic electroluminescence materials of present invention.

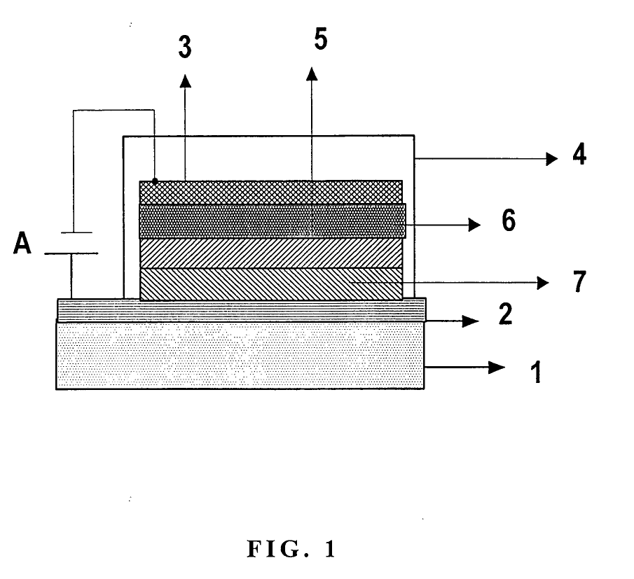

First, a 100 mm×100 mm glass substrate 1 is provided, then a 110 nm first electrode 2 (ITO) is plated on the glass substrate. After a 10 mm×10 mm luminescent area pattern is formed by Photo/Etching process, vacuum evaporation under a vacuum magnitude of 10⁻⁵ Pa is proceeded. At first, a 60 nm hole-transporting layer 7 of the material of hole-transporting is compound HT-4 in example 1 is formed under a evaporating rate of in 0.2 nm/sec. An organic luminescent layer 5 forms on hole-transporting layer 7 with the depth of 25 nm by co-vaporization with Alq3 and DCJTB, wherein that DCJTB is provided with content of 1.5 wt %. Then Alq3 as electron-transport 6 with the depth is 25 nm forms at a evaporating rate of 0.2 nm/sec. Finally, LiF (1.2 nm) and Al (150 nm) is formed for functioning as a second electrode 3 to complete the manufacturing of the organic functional layer. After the layers illustrated above are formed, the protecting layer 4 for air-tight membrane, which can cover the organic electroluminescent device completely in order to make sure the tightness, forms. The details of the structure are shown in FIG. 1.

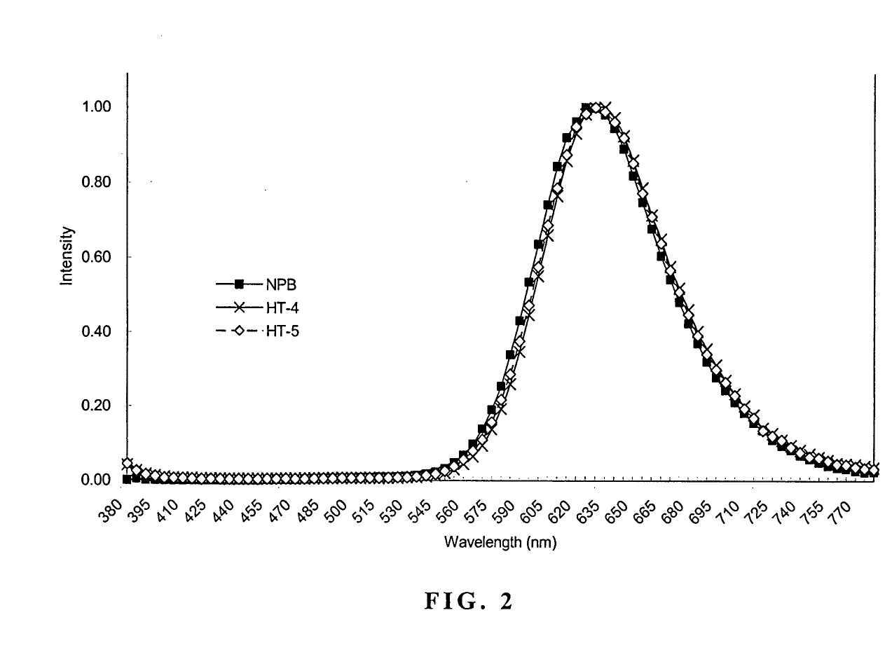

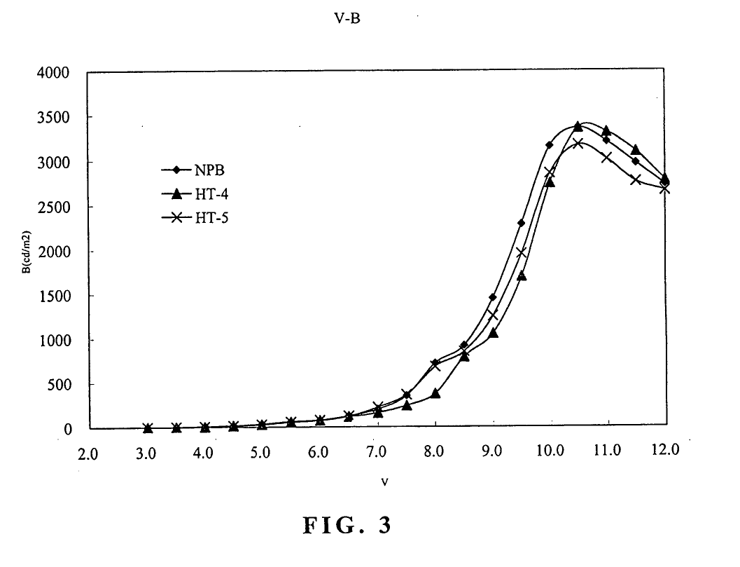

The applied current for driving the device illustrated can be direct current (DC), pulse current, or alternating current (AC). The measurement of luminescent property of the device is under DC driving apparatus herethrough Keithly 2000. The intensity of light is measured by photodiode array detector made by Otsuka Electronic Co. The result shows that he color of luminescence is red, CIE coordinates (0.63, 0.36), and the EL spectrum of the device is as shown in FIG. 2. Moreover, the result shows the wavelength of luminescent display is 360 nm. B-V is shown is FIG. 3, the brightness of 1447 cd/m² under 9V, and the efficiency is 1.24 cd/A.

### EXAMPLE 5

### The Device Comprises Hole-Transporting Material of HT-5

The device structure is as same as that in example 4 except that the hole-transporting material is replaced by HT-5. The measurement of luminescent property of the device is under DC driving. The result shows that the color of luminescence is red, the CIE coordinates are (0.64, 0.35), and EL spectrum of the device is as shown in FIG. 2. Moreover, the wavelength of luminescent display is 630 nm; B-V is shown is FIG. 3, the brightness of 1247 cd/m² under 9V, and the efficiency is 0.96 cd/A.

### EXAMPLE 6

### The Device Comprises Hole-Transporting Material of NPB

The device structure is as same as that in example 4 except that the hole-transporting material replaced by NPB. The measurement of luminescent property of the device is under DC driving. The results shows that the color of luminescence is red, the CIE coordinates are (0.64, 0.36), and the EL spectrum of the device is as shown in FIG. 2. Moreover, the wavelength of luminescent display is 630 nm; B-V is shown is FIG. 3, the brightness of 1447 cd/m² under 9V, and the efficiency is 1.27 cd/A.

### EXAMPLE 7

### Half-Life Examine

The device is operated under 9V, and the variation of brightness and time is recorded. The result is shown as follow:

| | Time(hrs) | | |
|---|---|---|---|
| | 96 | 192 | 264 |
| NPB | 12.5 | 25.0 | 31.3 |
| HT-4 | 9.1 | 18.2 | 27.3 |

Although the present invention has been explained in relation to its preferred embodiment, it is to be understood that many other possible modifications and variations can be made without departing from the spirit and scope of the invention as hereinafter claimed.

1. An organic electroluminescent material, comprising: a compound of formula (I) 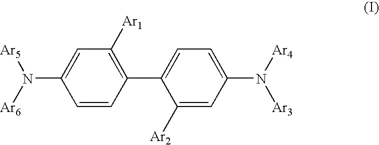
wherein
Ar₁, Ar₂, Ar₃, Ar₄, Ar₅and Ar₆ are independently hydrogen, substituted or unsubstituted C₁₋₆ alkyl, substituted or unsubstituted C₃₋₆ cycloalkyl, substituted or unsubstituted C₃₋₁₀ alkenyl, substituted or unsubstituted C₆₋₄₀ aromatic amino, substituted or unsubstituted C₆₋₄₀ aromatic, substituted or unsubstituted C₆₋₄₀ poly cyclic aromatic, or substituted or unsubstituted C₆₋₄₀ aralkyl.

2. An organic electroluminescent material of claim 1, wherein the compound of formula (I) is a material of hole-transporting layer (HTL).

3. An organic electroluminescent material of claim 1, wherein the substituted or unsubstituted C₁₋₆ alkyl is methyl, ethyl, propyl, isopropyl, butyl, isobutene, sec-butyl, tert-butyl, pentyl, or hexyl.

4. An organic electroluminescent material of claim 1, wherein the aromatic is provided with substituents, substituent is alkyl consisting of 1-6 carbon, cycloalkyl consisting of 3-6 carbon, alkoxy consisting of 1-6 carbon, aryloxy consisting of 5-18 carbon, aralkoxy consisting of 7-18 carbon, amino group substituted with C₅₋₁₆ aromatic, nitro, cyano, and C₁₋₆ ester or halogens.

5. An organic electroluminescent material of claim 1, wherein the C₃₋₆ cycloakoxy is cyclopropyl, cyclobutyl, cyclopentyl or cyclohexyl; C₁₋₆ alkoxy is methoxy, ethoxy, propoxy, isopropoxy, butoxy, isobutoxy, sec-butoxy, tert-butoxy, various isopropoxy or various hexoxyl; C₅₋₁₈ aryloxy is phenoxy, tolyoxymethyl or napthoxy; C₇₋₁₈ aralkoxy is phenyl ethoxyl, or naphthymethoxyl, substituted C₅₋₁₆ aromatic amino is diphenylamino, dimethylbenzeneamino, or naphthylphenylamino; C₁₋₆ ester is methoxycarbonyl, ethoxycarbonyl, or propoxycarbonyl; and halogens is fluorine, chlorine or bromine.

6. An organic electroluminescent material of claim 1, wherein the C₆₋₄₀ aromatic is phenyl, naphthyl, anthryl, phenanthryl, pyrenyl, biphenyl, terphenyl, triphenylamine, furan, thiophene or indole.

7. An organic electroluminescent material of claim 1, wherein the compound is selected from the group consisting of:

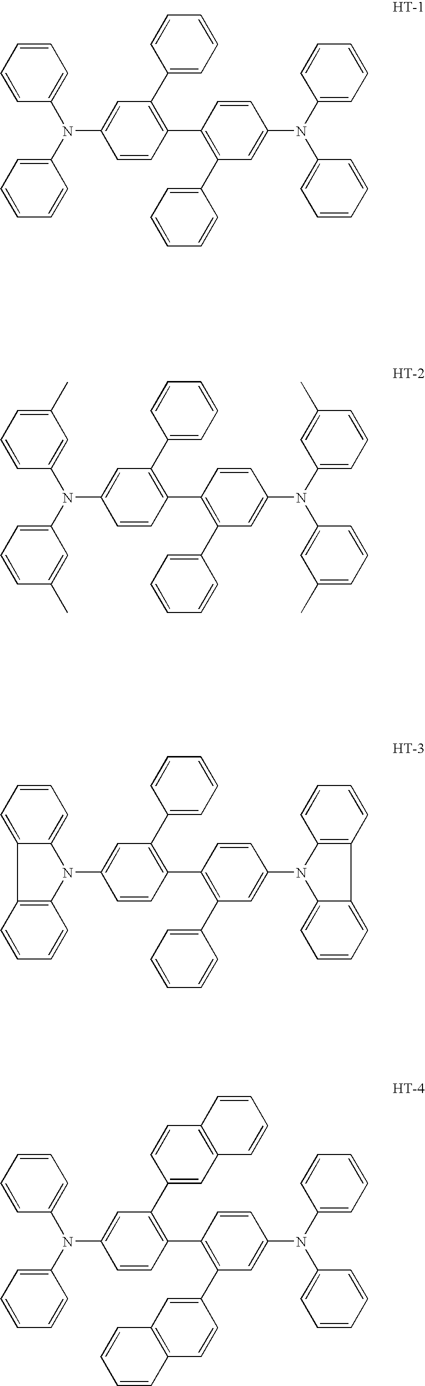

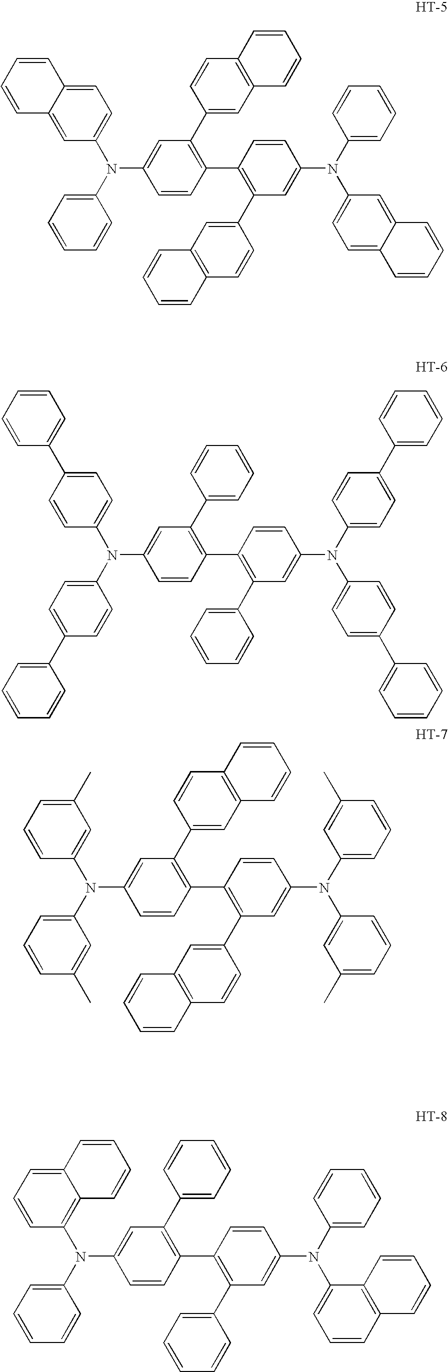

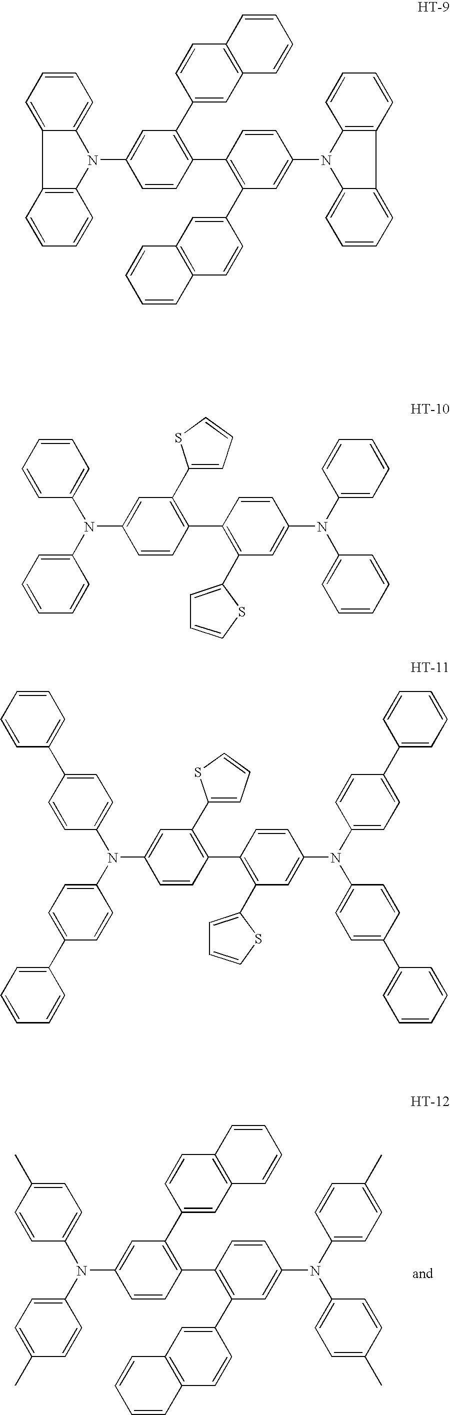

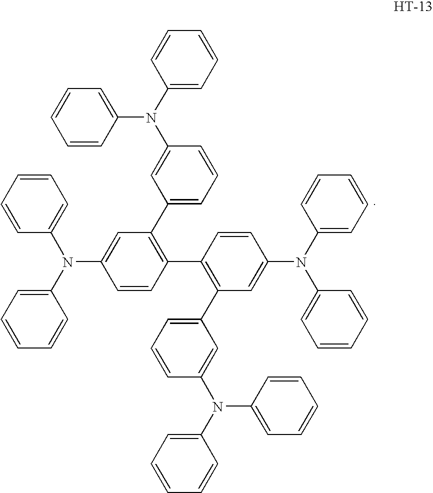

8. An organic electroluminescent device sequentially comprising: a first electrode, an organic functional layer and a second electrode disposed over a substrate;
the organic functional layer comprises a compound as formula (I): 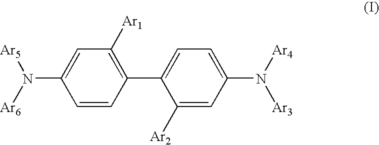
wherein
Ar₁, Ar₂, Ar₃, Ar₄, Ar₅ and Ar₆ are independently hydrogen, substituted or unsubstituted C₁₋₆ alkyl, substituted or unsubstituted C₃₋₆ cycloalkyl, substituted or unsubstituted C₃₋₁₀ alkenyl, substituted or unsubstituted C₆₋₄₀ aromatic amino, substituted or unsubstituted C₆₋₄₀ aromatic, substituted or unsubstituted C₆₋₄₀ poly cyclic aromatic, or substituted or unsubstituted C₆₋₄₀ aralkyl.

9. The organic electroluminescent device of claim 8, wherein the compound formula (I) is a material of hole-transporting layer (HTL).

10. The organic electroluminescent device of claim 8, wherein the substituted or unsubstituted C₁₋₆ alkyl is methyl, ethyl, propyl, isopropyl, butyl, isobutene, sec-butyl, tert-butyl, pentyl, or hexyl.

11. The organic electroluminescent device of claim 8, wherein the aromatic is provided with substituents, the substituent is alkyl consisting of 1-6 carbon, cycloalkyl consisting of 3-6 carbon, alkoxy consisting of 1-6 carbon, aryloxy consisting of 5-18 carbon, aralkoxy consisting of 7-18 carbon, amino group substituted with C₅₋₁₆ aromatic, nitro, cyano and C₁₋₆ ester or halogens.

12. The organic electroluminescent device of claim 8, wherein the C₃₋₆ cycloakoxy is cyclopropyl, cyclobutyl, cyclopentyl or cyclohexyl; C₁₋₆ alkoxy is methoxy, ethoxy, propoxy, isopropoxy, butoxy, isobutoxy, sec-butoxy, tert-butoxy, various isopropoxy or various hexoxyl; C₅₋₁₈ aryloxy is phenoxy, tolyoxymethyl or napthoxy; C₇₋₁₈ aralkoxy is phenyl ethoxyl, or naphthymethoxyl, substituted C₅₋₁₆ aromatic amino is diphenylamino, dimethylbenzeneamino, or naphthylphenylamino; C₁₋₆ ester is methoxycarbonyl, ethoxycarbonyl, or propoxycarbonyl; and halogens is fluorine, chlorine or bromine.

13. The organic electroluminescent device of claim 8, wherein the C₆₋₄₀ aromatic is phenyl, naphthyl, anthryl, phenanthryl, pyrenyl, biphenyl, terphenyl, triphenylamine, furan, thiophene or indole.

14. The organic electroluminescent device of claim 8, wherein organic material of an organic functional layer is selected from the group consisting of

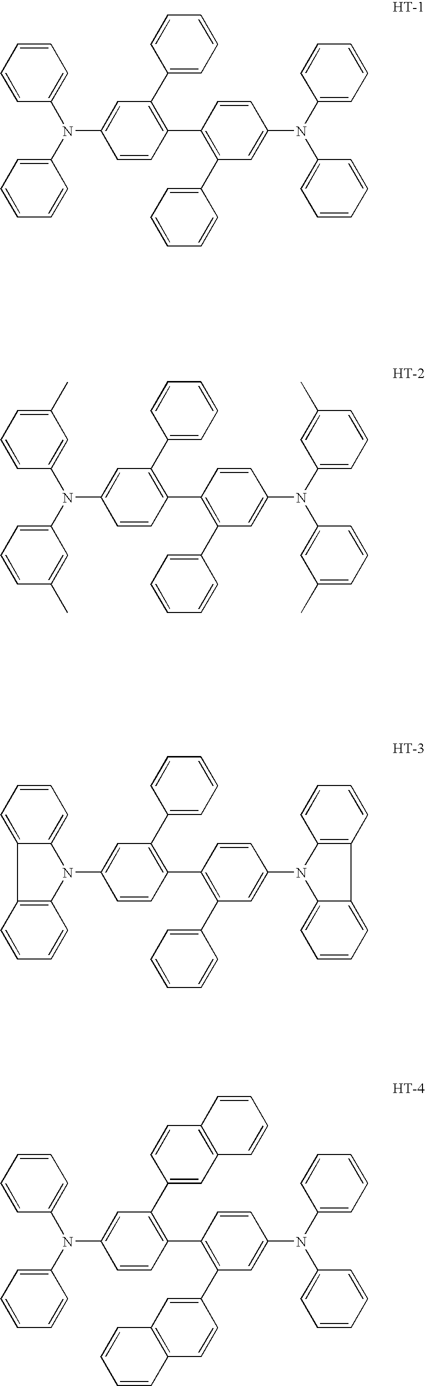

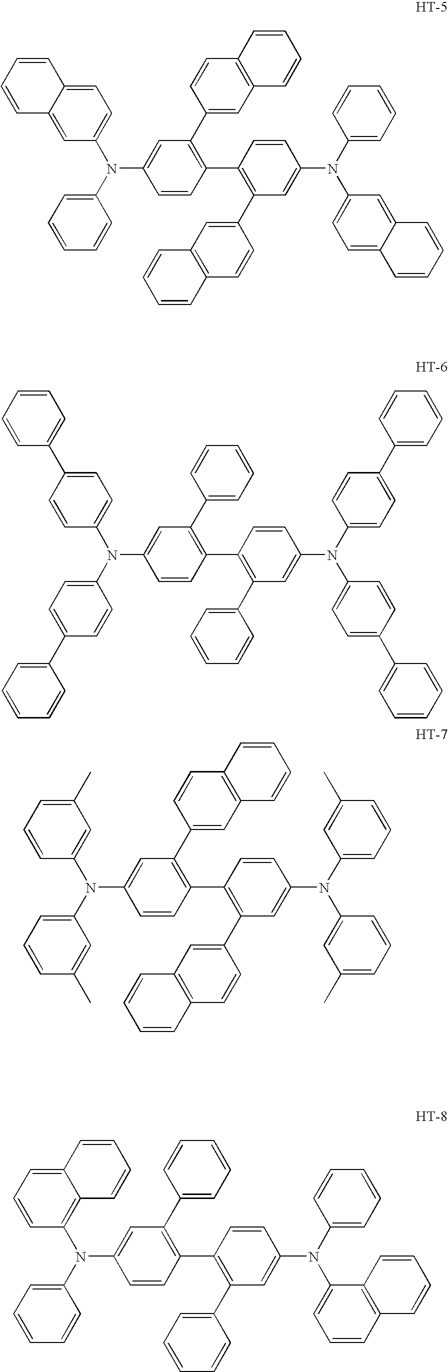

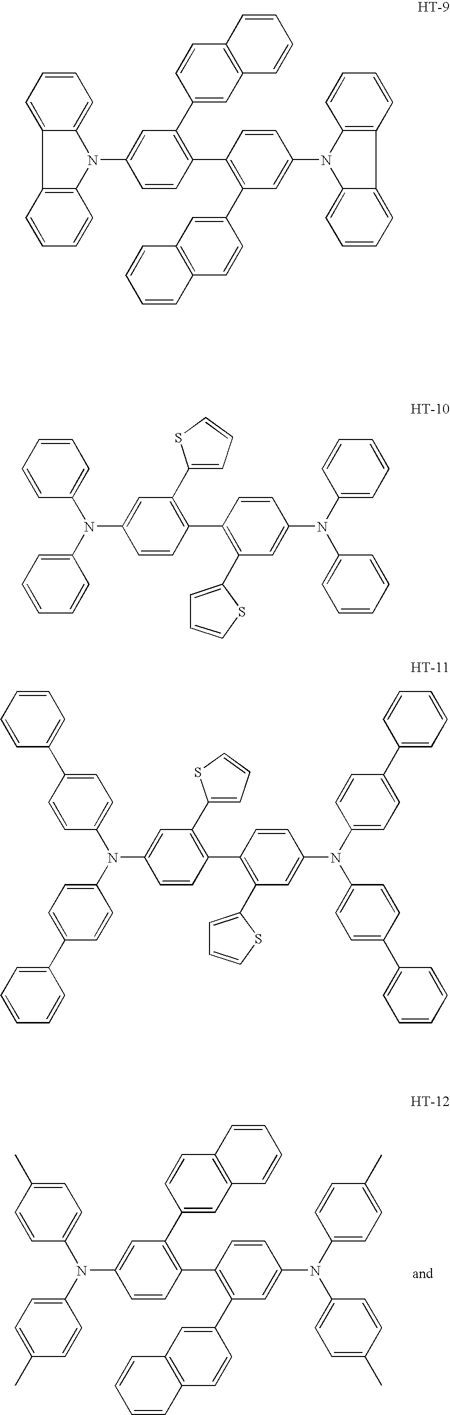

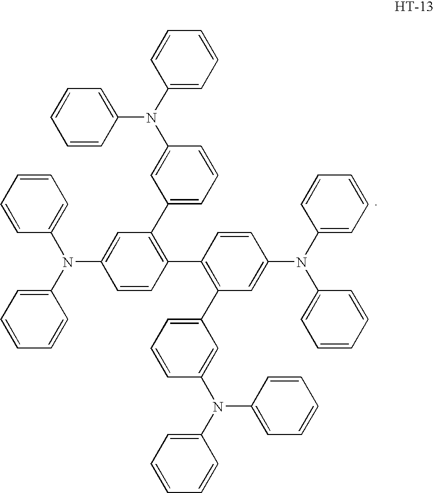

15. The organic electroluminescent device of claim 8, wherein the substrate is a transparent substrate.

16. The organic electroluminescent device of claim 8, wherein the first electrode is a transparent electrode.

17. The organic electroluminescent device of claim 8, wherein the organic functional layer is a multi-layered laminate sandwiched between the first electrode and the second electrode.

18. The organic electroluminescent device of claim 17, wherein the multi-layered laminate comprises a hole-injectioning layer, a hole-transporting layer, a luminescence layer, an electron-injectioning layer or a combination thereof.

19. The organic electroluminescent device of claim 17, wherein the thickness of the multi-layered laminate is 1 nm-1 μm.

20. The organic electroluminescent device of claim 17, wherein the multi-layered laminate is formed on the first electrode by evaporation, spin-coating, ink jet printing or printing.

21. The organic electroluminescent device of claim 8, wherein the organic electroluminescent material is formed by vacuum deposition, MBE, dip coating, spin-coating casting, bar code or roll coating.

22. The organic electroluminescent device of claim 8, wherein the second electrode is formed by evaporation, E-gun or sputtering.

23. The organic electroluminescent device of claim 8, wherein the second electrode is made of aluminum, Al—Li alloy, calcium, Ag—Mg alloy, Ag alloy or Ag.
